(19) United States
(12) Patent Application Publication
(10) Pub. No.: US 2021/0077554 A1
(43) Pub. Date: Mar. 18, 2021

(54) Methods of Neoplasm Treatment Utilizing Complementary Oncolytic Viruses and CAR T-Cells

(71) Applicant: The Board of Trustees of the Leland Stanford Junior University, Stanford, CA (US)

(72) Inventors: Amin Aalipour, Stanford, CA (US); Sanjiv S. Gambhir, Stanford, CA (US)

(73) Assignee: The Board of Trustees of the Leland Stanford Junior University, Stanford, CA (US)

(21) Appl. No.: 17/020,670

(22) Filed: Sep. 14, 2020

Related U.S. Application Data
(60) Provisional application No. 62/900,348, filed on Sep. 13, 2019.

(51) Int. Cl. A61K 35/768; C12N 7/00; A61K 35/17

(52) U.S. Cl. CPC A61K 35/768; A61K 9/0019; A61K 35/17; C12N 7/00

(57) ABSTRACT

Methods of treatment utilizing complimentary transgenic oncolytic virus and engineered CAR T-cells are provided. Oncolytic viruses are engineered such that they can express an ectopic antigen on neoplastic cells. CAR T-cells are engineered to recognize the ectopic antigen are utilized in various methods of treatment. Accordingly, a transgenic oncolytic virus can be administered to a neoplasm to induce expression of an ectopic antigen and then that neoplasm can be treated with a complimentary CAR T cells engineered to recognize the ectopic antigen to induce an immune response against the neoplasm, resulting in neoplastic cell death and/or clearance.

### CROSS REFERENCE TO RELATED APPLICATIONS

This application claims priority to U.S. Provisional Application Ser. No. 62/900,348, entitled “Methods of Neoplasm Treatment Utilizing Oncolytic Viruses and CAR T-Cells” by Amin Aalipour et al., filed Sep. 13, 2019, which is incorporated herein by reference in its entirety.

### TECHNICAL FIELD

The invention is generally directed to methods involving treatments of neoplasms, and more specifically treatments of neoplasms using complementary transgenic oncolytic viruses and genetically engineered CAR T cells.

### BACKGROUND

T cells are a type of immune cell that has an important role in immune responses against foreign invaders such as viruses and unhealthy host cells such as cancer cells. T cells are distinguished from other immune cells by the presence of the T-cell receptor that is present on the cell surface. T cells have many important functions, including identifying infected or cancer cells, stimulating immune responses against infected or cancer cells, and killing or removing infected or cancer cells.

Chimeric antigen receptor (CAR) T cells are T cells that have been engineered to produce an artificial T-cell receptor on their cell surface. The engineered CAR is designed to specifically recognize a protein or receptor that is present on surface of a cell. When a CAR T cell recognizes the protein or receptor, it stimulates an immune response against that cell, which can specifically kill and/or clear that cell. A number of particular CAR T cells have been engineered to recognize cancer cells. For instance, ongoing clinical trials are investigating the therapeutic benefit of CAR T cells to recognize the CD19 receptor for the treatment of various B-cell lymphomas.

### SUMMARY

Various embodiments are directed towards methods treatments for cancer utilizing complementary oncolytic virus and CAR T cells. In various embodiments, T cells are engineered to recognize a particular ectopic antigen and oncolytic viruses are engineered to induce expression of the ectopic antigen in cancer cells. In various embodiments, an individual with cancer is treated with an oncolytic virus to induce expression of the ectopic antigen within the cancer and then further treated with CAR T cells to target the cancer. In various embodiments, the CAR T cells utilized to treat the individual are extracted from the individual, then engineered to express the CAR recognizing the ectopic protein, and then transplanted back into the individual for treatment.

In an embodiment, an individual having a neoplasm is treated. An individual is administered an oncolytic virus capable of selectively expressing an ectopic antigen on the surface of neoplastic cells. The individual is administered genetically engineered T cells that express a complementary chimeric antigen receptor that recognizes the antigen.

In another embodiment, the T cells are autologous.

In yet another embodiment, T cell are harvested from the individual. The T cells are genetically engineered to express the complimentary chimeric antigen receptor that recognizes the antigen.

In a further embodiment, the T cells are genetically engineered by viral vector transduction or site-directed mutagenesis.

In still yet another embodiment, the T cells are further genetically modified to enhance T cell function.

In yet a further embodiment, the ectopic antigen is an exogenous antigen.

In an even further embodiment, the ectopic antigen is an endogenous antigen that is expressed by the neoplastic cells.

In yet an even further embodiment, the ectopic antigen is GD2, mesothelin, BCMA, PSMA, EGFRvIII, MUC1, or NY-ESO-1.

In still yet an even further embodiment, the antigen is CD19.

In still yet an even further embodiment, the oncolytic virus is derived from reovirus, Seneca Valley virus (SVV), vesicular stomatitis virus (VSV), Newcastle disease virus (NDV), herpes simplex virus (HSV), measles virus, adenovirus, or poxvirus.

In still yet an even further embodiment, the oncolytic virus is derived from vaccinia virus.

In still yet an even further embodiment, expression of Thymidine Kinase by the vaccinia is disrupted.

In still yet an even further embodiment, the oncolytic virus is parenterally administered.

In still yet an even further embodiment, the oncolytic virus is intratumorally administered.

In still yet an even further embodiment, the engineered T-cells are administered via intravenous, intra-arterial, or intralymphatic delivery.

In still yet an even further embodiment, the treatment is further combined with surgery, immunotherapy, chemotherapy, radiation therapy, targeted therapy, hormone therapy, stem cell therapies, or blood transfusions.

In still yet an even further embodiment, the individual is administered a chemotherapeutic agent.

In still yet an even further embodiment, the administration of the oncolytic virus and the administration of the genetically engineered T cells is performed as part of an adjuvant or a neoadjuvant treatment.

In still yet an even further embodiment, the neoplasm is anal cancer, astrocytomas, basal cell carcinoma, bile duct cancer, bladder cancer, breast cancer, breast adenocarcinoma (BRCA), cervical cancer, chronic myeloproliferative neoplasms, colorectal cancer, endometrial cancer, ependymoma, esophageal cancer, diffuse large B-cell lymphoma (DLBCL), esthesioneuroblastoma, Ewing sarcoma, fallopian tube cancer, gallbladder cancer, gastric cancer, gastrointestinal carcinoid tumor, hepatocellular cancer, hypopharyngeal cancer, Kaposi sarcoma, Kidney cancer, Langerhans cell histiocytosis, laryngeal cancer, liver cancer, lung cancer, melanoma, Merkel cell cancer, mesothelioma, mouth cancer, neuroblastoma, non-small cell lung cancer, osteosarcoma, ovarian cancer, pancreatic cancer, pancreatic neuroendocrine tumors, pharyngeal cancer, pituitary tumor, prostate cancer, rectal cancer, renal cell cancer, retinoblastoma, skin cancer, small cell lung cancer, small intestine cancer, squamous neck cancer, testicular cancer, thymoma, thyroid cancer, uterine cancer, vaginal cancer, or vascular tumors.

In an embodiment, a kit is for treating a neoplasm. The kit includes an oncolytic virus that expresses an ectopic antigen. The kit includes an expression vector for genetically engineering T cells to express a complementary chimeric antigen receptor capable of recognizing the ectopic antigen.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The description and claims will be more fully understood with reference to the following figures and data graphs, which are presented as exemplary embodiments of the invention and should not be construed as a complete recitation of the scope of the invention.

FIG. 1 provides a schematic of a method to treat an individual via tumor-selective delivery of CAR targets by an oncolytic virus followed by selective clearance by matched CAR T-cells in accordance with an embodiment of the invention.

FIG. 2 provides data graphs of B16 mouse melanoma cell line expressing TurboRFP and mCD19, utilized in accordance with various embodiments.

FIG. 3 provides data graphs showing mCD19 CAR T-cells are not cytotoxic against B16 cells in co-culture but exhibit dose-dependent activity against a B16 cell line engineered to express mCD19, generated in accordance with various embodiments. Mock T-cells do not exhibit activity against either cell line. 24 hour E:T=4 p<0.0001, F=49.23, R square=0.9486 by ANOVA. 48 hour E:T=4 p<0.0001, F=49.65, R square=0.9490 by ANOVA. n=3 independent cultures for each combination, E:T ratio, and time point.

FIG. 4 provides data graphs showing low- and high-mCD19-expressing B16 cell lines (left) exhibit antigen density-dependent susceptibility to mCD19 CAR T-cells (right) at 24 hours of co-culture, generated in accordance with various embodiments (n=5 independent cultures with each cell line, p=0.0116, t=3.258, df=8 by two-tailed unpaired t-test).

FIG. 5 provides cytometry florescent peaks depicting that CD69 is only upregulated in antigen-matched co-cultures for both CD4 and CD8 T-cells, generated in accordance with various embodiments.

FIG. 6 provides data graphs depicting tumor volume (left) and survivability (right) of mice with B16 cancer cells treated with CAR T cells, generated in accordance with various embodiments. mCD19 CAR T-cells significantly delay B16-mCD19 tumor progression in vivo and confer a survival benefit relative to antigen-mismatched therapy groups. Day 8 tumor volume p<0.0001, F=19.14, R square=0.7322 by ANOVA. Kaplan-Meier survival curve p=0.0011, df=2, Chi square=13.58 by Mantel-Cox test. Number of independent mice in each group as follows: n=5 (B16+CAR), n=6 (B16-mCD19+Mock), n=6 (B16-mCD19+CAR). Data is shown as mean±standard error of the mean (s.e.m). * indicates statistical significance at p<0.05.

FIG. 7 provides a schematic of oncolytic vaccinia virus expression construct (top) and data graphs of oncolytic vaccinia virus transduction in B16 cells, utilized in accordance with various embodiments. Both a control and mCD19 oncolytic vaccinia virus were generated by insertion of transgenes into the viral thymidine kinase locus. mCD19 VV induces time- and dose-dependent expression of Fluc (left panel) and exhibits lytic activity (middle-left panel) with the B16 cell line after 48 hours of culture at an MOI of 1. mCD19 VV also induces time- and dose-dependent expression of YFP and mCD19 with the B16 cell line. Data is shown as mean±s.e.m. pE/L, early/late promoter; p7.5, vaccinia 7.5 kD early promoter; pLEO, late-early optimized promoter; GPT, guanine phosphoribosyltransferase.

FIG. 8 provides data graphs depicting In vitro co-culture studies with B16 (top) and SB28 (bottom left) highlight the greatest combinatorial toxicity with co-culture of mCD19 CAR T-cells and tumor cells infected with mCD19 VV, generated in accordance with various embodiments. Mock+CD19 VV vs. CAR+CD19 VV: p<0.0001, t=9.413, df=10 (B16, 24 hours); p=0.0009; p=0.0009, t=4.654, df=10 (B16, 48 hours); p<0.0001, t=6.993, df=10 (SB28, 24 hours); p=0.0073, t=3.356, df=10 (SB28 48 hours). CAR+control VV vs. CAR+CD19 VV: p=0.0272, t=2.584, df=10 (B16, 24 hours); p=0.0337, t=2.459, df=10 (B16, 48 hours); p=0.0076, t=3.332, df=10 (SB28, 24 hours); p=0.0377, t=2.395, df=10 (SB28, 48 hours). n=6 independent cultures for each combination, timepoint, and cell line. CAR T-cell efficacy also benefits from VV-induced augmentation in levels of mCD19 when co-cultured with a B16-mCD19 cell line that expresses low levels of mCD19 (bottom right). CAR+control VV vs. CAR+CD19 VV: n=5 independent cultures for each combination, p=0.0618, t=2.170, df=8.

FIG. 9 provides cytometry florescent peaks depicting that the antigen-matched combination of mCD19 VV infected B16 cells and mCD19 CAR T-cells in vitro leads to upregulation of the early activation marker CD69 on both CD4 and CD8 T-cells at 24 and 48 hours of co-culture, generated in accordance with various embodiments.

FIG. 10 provides cytometry population results depicting the presence of CAR T-cells eliminates all mCD19+ cells by 48 hours of co-culture with mCD19 VV-infected B16 cells, generated in accordance with various embodiments. Populations shown gated on CD4-CD8-double negative cells. All statistical analysis performed with unpaired two-tailed t-tests. Data is shown as mean±s.e.m. * indicates statistical significance at p<0.05, ** indicates statistical significance at p<0.01, **** indicates statistical significance at p<0.0001.

FIG. 11 provides data graphs depicting that intratumorally administered VV can selectively deliver both mCD19 and YFP to B16 cells in an immunocompetent, orthotopic model of melanoma with minimal infection of intratumoral T-cells, generated in accordance with various embodiments. Rates of infection can be increased with a single dose of 5 Gy TBI on the day of the first virus injection. % mCD19+ TBI (n=2 independent mice) vs. no TBI (n=3 independent mice): p=0.0144, t=5.126, df=3. % YFP+ TBI vs. no TBI: p=0.008, t=6.332, df=3. Data is shown as mean±s.e.m in. * indicates statistical significance at p<0.05, ** indicates statistical significance at p<0.01.

FIG. 12 provides data graphs depicting tumor volume of mice intratumorally administered VV, generated in accordance with various embodiments. Control (n=5 independent mice) and mCD19 VV (n=4 independent mice) can yield modest delays in B16 tumor progression relative to mice only receiving TBI (n=6 independent mice). TBI Only vs. Control VV: day 11 tumor volume p=0.0040, t=3.836, df=9. TBI Only vs. CD19 VV: day 11 tumor volume p=0.0246, t=2.762, df=8. The two viruses also have a similar therapeutic index in vivo Control VV vs. CD19 VV: p=0.7293, t=0.3602, df=7.

FIG. 13 provides data graphs depicting tumor volume (left, middle) and survivability (right) of mice with B16 cancer cells treated with intratumorally administered VV and CAR T cells, generated in accordance with various embodiments. The combination of mCD19 VV together with mCD19 CAR T-cells leads to the most significant delay in B16 tumor progression relative to all other combinations of single and double agents. Fold change in tumor volume (left) and tumor volume on day 11 (middle) shown. Mock+control VV (n=4 independent mice) vs. CAR+CD19 VV (n=5 independent mice): day 11 tumor volume p=0.006, t=3.885, df=7. Mock+CD19 VV (n=5 independent mice) vs. CAR+CD19 VV: day 11 tumor volume p=0.0031, t=4.183, df=8 CAR+control VV (n=4 independent mice) vs. CAR+CD19 VV: day 11 tumor volume p=0.0051, t=4.006, df=7. The delay in tumor progression is also associated with a survival benefit for mice treated with the antigen-matched therapy combination (right). Kaplan-Meier survival curve p=0.0003, df=5, Chi square=23.33 by Mantel-Cox test. Number of independent mice in each group as follows: n=4 (Mock Only), n=4 (Ctrl VV Only), n=5 (Mock+Ctrl VV), n=5 (Mock+CD19 VV), n=4 (CAR+Ctrl VV), n=5 (CAR+CD19 VV). Non-survival statistical analysis performed with unpaired two-tailed t-tests. Data is shown as mean±standard deviation in. * indicates statistical significance at p<0.05, ** indicates statistical significance at p<0.01.

FIG. 14 provides cytometry florescent peaks depicting analysis of intratumoral T-cells revealing upregulation of activation markers CD25 and OX40 on both CD4 and CD8 T-cells in the antigen-matched combination group, generated in accordance with various embodiments. Data is shown as mean±s.e.m in (A) and mean±standard deviation in (C). * indicates statistical significance at p<0.05, ** indicates statistical significance at p<0.01.

FIG. 15 provides cytometry population results depicting transduction efficiency of T cells to induce CAR expression as measured by Protein L staining, generated in accordance with various embodiments.

### DETAILED DESCRIPTION

Turning now to the drawings and data, systems and methods for treating neoplasms and cancers utilizing complementary oncolytic virus and chimeric antigen receptor (CAR) T cells are provided, in accordance with various embodiments. In several embodiments, an oncolytic virus is engineered to induce transgenic expression of an ectopic antigen on the surface of a neoplastic and/or a cancer cell. In several embodiments, T cells are engineered to express a CAR that is capable of recognizing the ectopically expressed antigen. In several embodiments, an individual with a cancer or neoplasm is treated by administering to the individual an oncolytic virus to induce expression of an ectopic antigen on the surface of the cancer or neoplastic cells; the individual is further administered complimentary CAR T cells that recognize the ectopic antigen, which can induce an immune response against the cancer or neoplastic cell. In several embodiments, an individual's own T cells are engineered to express a CAR capable of recognizing a particular antigen to induce an immune response. Further, in several embodiments, to engineer an individual's own CAR T cells, T cells are harvested from the individual and CAR expression is induced into the T cell, which can subsequently be returned to the individual as part of a treatment. In several embodiments, further treatments may be performed on the individual based on the individual's particular cancer or neoplasm, such as (for example) surgery, immunotherapy, chemotherapy, radiation therapy, targeted therapy, hormone therapy, stem cell therapies, and blood transfusions.

Generally, the various embodiments described herein provide a number of benefits within the realm of neoplasm and cancer treatments, especially neoplasms and cancers that lack an easily targetable, cancer-limited, and homogeneously expressed endogenous protein or receptor. The promise of CAR T cell therapy has been demonstrated within various B-cell lymphomas that have high endogenous expression of the receptor CD19. Further, the high expression of CD19 in cancerous B cells is rather specific to B cells and thus CAR T-cell therapy targeting CD19 does not have many side effects on cells other than B cells and cells not dependent on B cells for function. The high expression and specificity of CD19 renders B cell lymphomas an easily targetable cancer for a CAR T-cell therapy. Unfortunately, many other cancers, especially solid tumor cancers, lack an endogenous CAR T-cell therapy target that is highly expressed and specific.

In accordance with various embodiments described herein, it is a goal to either amplify expression of an endogenous target antigen or, in some instances, introduce expression of an exogenous target antigen on the cell surface of a neoplastic or cancer cell, such that the endogenous or exogenous target antigen is highly expressed. Accordingly, in various embodiments, a transgenic oncolytic virus is utilized to infect neoplastic cells to induce high expression levels of an ectopic CAR T-cell therapy target antigen. In various embodiments, T cells are engineered to express a complimentary CAR that specifically recognizes the target antigen. In various embodiments, the complimentary engineered CAR T cells are delivered to neoplastic cells with induced ectopic expression of the target, which can activate an immune response against the neoplastic cells. In various embodiments, complimentary oncolytic virus and CAR T cells are utilized in a treatment for an individual's neoplasm or cancer.

Throughout the description, the terms neoplasm and cancer (or neoplastic cell and cancer cell) are utilized interchangeably. A neoplasm, as understood in the field, is a new and abnormal growth of tissue, and thus includes benign growths (e.g., benign tumors) and cancerous growths. Similarly, a cancer is an abnormal growth of cells with the potential to metastasize and to spread to other areas of the body. Accordingly, the various embodiments described herein can be applied to neoplasms and cancers, unless specified to be exclusive to one or the other.

The term oncolytic virus is utilized to describe viruses or viral vectors that preferentially infect or transduce neoplastic cells. In some instances, infection or transduction of a neoplastic cells with oncolytic virus can result in the lysis and/or death the cell, but lysis and/or death is not necessarily required. In various embodiments described herein, an oncolytic virus is engineered to induce ectopic expression of a CAR T cell target antigen. A number of modified viruses or viral vectors can be utilized as oncolytic viruses, including (but not limited to) reovirus, Seneca Valley virus (SVV), vesicular stomatitis virus (VSV), Newcastle disease virus (NDV), herpes simplex virus (HSV), measles virus, adenovirus, and poxvirus (e.g., vaccinia virus).

### Treatment Utilizing Oncolytic Virus and CAR-T Cells

A number of embodiments utilize a complimentary approach of administering an oncolytic virus to deliver an ectopic surface antigen to a neoplastic cell and administering CAR T cell that targets the ectopic antigen. In many embodiments, the oncolytic virus is genetically engineered with an expression cassette to induce expression of an ectopic antigen in a neoplastic cell that is infected by the oncolytic virus. In many embodiments, T cells are genetically engineered express a CAR that is capable of recognizing the ectopic antigen.

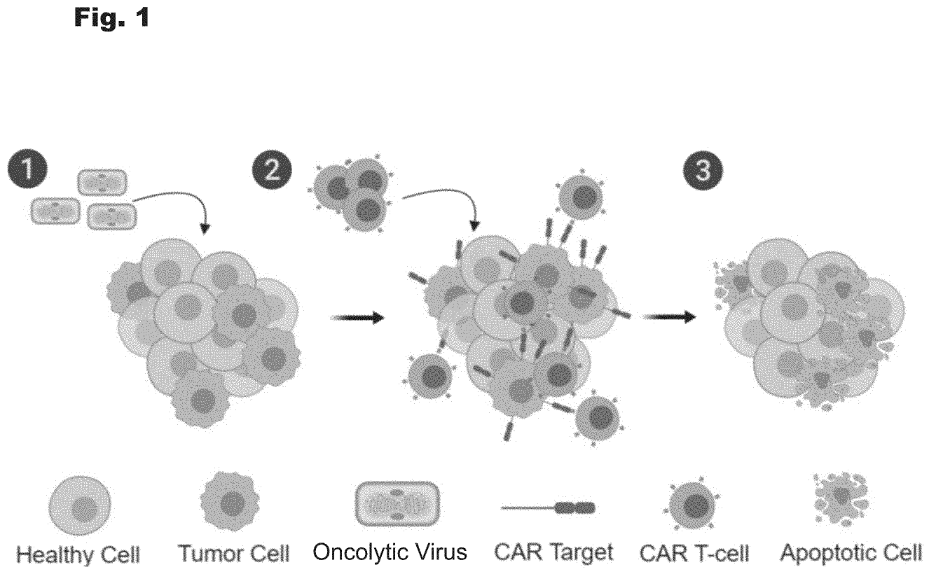

An overview of strategy for CAR T-cell therapy in accordance with an embodiment is provided in FIG. 1. As shown in step 1, a transgene expressing oncolytic virus is administered to a tissue. In many instances, unhealthy neoplastic cells (e.g., tumor cells) are mixed within and/or bordering healthy cells (i.e., non-neoplastic). To specifically target the unhealthy cells, the engineered virus has specificity to infect and/or replicate within neoplastic cells, leaving the healthy cells intact. The transgenic oncolytic virus includes an expression vector to induce transgenic expression of an ectopic antigen (i.e., CAR target) specifically within the tumor cells. The expression of the CAR target is shown on the tumor cells in step 2.

As shown in step 2, a complimentary CAR T cell is administered to the tissue. The CAR T cells can be administered concurrently with the administration of the oncolytic virus and/or soon before/after. The CAR T cells have been genetically engineered with an expression cassette to induce expression of a complimentary CAR to be produced on the cell surface of the T cell that can target the ectopic antigen expressed on the tumor cells. As shown in FIG. 1, the engineered CAR T cells specifically target and interact with the unhealthy neoplastic cells via the ectopic antigen, which can result in an immune response against these cells. As shown in step 3, as result of the oncolytic virus infection and the induced immune response via the CAR T cells, the unhealthy cells apoptose, lyse, and/or are cleared, leaving behind the healthy cells.

Any appropriate oncolytic virus can be utilized to deliver transgene expression to neoplastic cells. In some embodiments, an oncolytic virus is engineered to achieve desired attributes, which may include cellular tropism, virus attenuation, and enhanced transgene expression. In various embodiments, an oncolytic virus is derived from reovirus, Seneca Valley virus (SVV), vesicular stomatitis virus (VSV), Newcastle disease virus (NDV), herpes simplex virus (HSV), measles virus, adenovirus, and/or poxvirus (e.g., vaccinia virus).

In some embodiments, the tropism of an oncolytic virus is engineered such that it specifically targets neoplastic cells. Various techniques can be utilized to achieve desired tropism, such as (for example) direct evolution in which viruses are screened and selected for their ability to be highly active within neoplastic cells. In some embodiments, viruses are engineered to express a particular protein involved in virus infection or transduction that preferentially interacts with receptors present on particular cell type (e.g., HER2+ breast cancer cells), resulting in preferential infection or transduction. In some embodiments, viruses are engineered to have preferential molecular activity and/or preferential replication within a particular cell type. For example, non-dividing and lowly dividing cells have low expression of thymidine kinase (TK) but cells undergoing rapid division (e.g., neoplastic cells) have high expression of thymidine kinase. HSV and vaccinia virus include a TK gene within its genome to promote its molecular activity and replication in all cell types. To reduce HSV and vaccinia virus tropism in non-dividing and lowly dividing cells, and thus to increase tropism within neoplastic cells, the TK gene can be deleted and/or altered, rendering virus activity ineffective in non-dividing and lowly dividing cells.

In some embodiments, an oncolytic virus is attenuated such that it is a weakened virulence. In some embodiments, particular components of a viral genome (e.g., viral genes) are deleted and/or altered such that the oncolytic virus is less capable of replicating and/or stimulating a particular viral-induced response as compared to the wild-type. For example, deleting and/or altering the TK gene also attenuates the virulence of HSV and vaccinia viruses.

In some embodiments, one or more transgenes are inserted within an oncolytic virus genome to be expressed within neoplastic cells. Accordingly, in some embodiments, an oncolytic virus genome includes an expression cassette having an operably linked regulatory sequence to promote expression of one or more a transgenes and an operably linked poly A signal. In some embodiments, a transgene has its own operably linked regulatory sequence and poly A signal. In some embodiments, a plurality of transgenes share an operably linked regulatory sequence and poly A signal. When a plurality of transgenes share an operably linked regulatory elements, an internal ribosome entry site (IRES) or self-cleaving peptide sequence (e.g., 2A peptide) can be utilized to generate individual transgene products from the shared expression cassette. Alternatively, a plurality of transgenes can be fused to form one long peptide to be expressed as a singular product.

In some embodiments, at least one transgene is expressed within a neoplastic cell to produce an ectopic CAR T-cell antigen. In some embodiments, an ectopic CAR T-cell antigen is exogenous, meaning the antigen is a gene sequence from a different species. In some embodiments, an ectopic CAR T-cell antigen is an endogenous antigen, such as one typically expressed on a tumor cell. An oncolytic virus can be utilized to bolster expression of an endogenous antigen on a neoplastic cell, thereby improving CAR T-cell recognition of the neoplastic cell. In some embodiments, an endogenous antigen is GD2, mesothelin, BCMA, PSMA, EGFRvIII, MUC1, or NY-ESO-1.

In some embodiments, T cells are genetically engineered to express a particular CAR capable of targeting a particular antigen. Any appropriate means to obtain T cells to be engineered can be utilized. In some embodiments, T cells to be engineered are autologous, meaning the T cells are derived from the patient to be treated. In some embodiments, T cells to be engineered are allogeneic, which can be derived from a healthy donor and/or derived from cultivated in vitro cell lines. In some embodiments, derived T cells are expanded for genetic engineering. Any appropriate genetic engineering can be utilized, including (but not limited to) viral vector transduction (e.g., lentiviral vector) and site-directed mutagenesis (e.g., CRISPR mutagenesis).

Oncolytic viruses and engineered T cells can be administered to an individual in accordance with any appropriate treatment regime. In some embodiments, an oncolytic virus is administered to reach the site of neoplastic cell growth. In some embodiments, an oncolytic virus is administered via an intratumoral delivery. In various embodiments, an oncolytic virus is administered via an enteral or a parenteral delivery, such as an intravenous, an intramuscular, or a subdermal delivery. The oncolytic virus may be dispersed in a pharmaceutically acceptable formulation for injection. In various embodiments, an individual is administered and order of 10⁵, 10⁶, 10⁷, 10⁸, 10⁹, 10¹⁰, 10¹¹, 10¹², or 10¹³ pfu of virus. In various embodiments, an individual is administered an oncolytic virus multiple times, including 1, 2, 3, 4, 5, 6, or more times. In some embodiments, the T cells are administered via intravenous, intra-arterial, or intralymphatic delivery.

### Pharmaceutical Formulations and Treatments

Various embodiments are directed to treatments based on an oncolytic virus and CAR T cell administration. As described herein, oncolytic viruses can be engineered to express an antigen and an individual's T cells can be engineered to recognize that antigen. In various embodiments, oncolytic viruses CAR T cells are administered to an individual having a neoplasm.

In accordance with this disclosure, the term “pharmaceutical composition” relates to a composition for administration to an individual. In some embodiments, a pharmaceutical composition comprises oncolytic virus for enteral or a parenteral administration, or for direct injection into a neoplasm. In some embodiments, a pharmaceutical composition comprising is administered to the individual via infusion or injection.

In some embodiments, oncolytic virus and/or CAR T cells are administered in a therapeutically effective amount as part of a course of treatment. As used in this context, to “treat” means to ameliorate at least one symptom of the disorder to be treated or to provide a beneficial physiological effect. For example, one such amelioration of a symptom could be reduction of tumor size.

A therapeutically effective amount can be an amount sufficient to prevent reduce, ameliorate or eliminate the symptoms of cancer. In some embodiments, a therapeutically effective amount is an amount sufficient to reduce the growth of neoplasm and/or metastasis of a cancer.

### Cancer Treatments

A number of embodiments are directed towards treating an individual for a neoplasm and/or cancer. Accordingly, an embodiment to treat an individual is as follows:
(i) extract T-cells from the individual
(ii) engineer the individual's T-cells to express chimeric antigen receptors (CARs) to target an ectopic antigen
(iii) administer transgene expressing oncolytic virus to the individual to induce expression of the ectopic antigen on neoplastic cells
(iv) administer the engineered CAR T cells to the individual

In some embodiments, the treatment is an adjuvant treatment. In some embodiments, the treatment is a neoadjuvant treatment.

In accordance with various embodiments, numerous types of neoplasms can be treated, especially solid tumors. Neoplasms that can be treated include (but not limited to) anal cancer, astrocytomas, basal cell carcinoma, bile duct cancer, bladder cancer, breast cancer, breast adenocarcinoma (BRCA), cervical cancer, chronic myeloproliferative neoplasms, colorectal cancer, endometrial cancer, ependymoma, esophageal cancer, diffuse large B-cell lymphoma (DLBCL), esthesioneuroblastoma, Ewing sarcoma, fallopian tube cancer, gallbladder cancer, gastric cancer, gastrointestinal carcinoid tumor, hepatocellular cancer, hypopharyngeal cancer, Kaposi sarcoma, Kidney cancer, Langerhans cell histiocytosis, laryngeal cancer, liver cancer, lung cancer, melanoma, Merkel cell cancer, mesothelioma, mouth cancer, neuroblastoma, non-small cell lung cancer, osteosarcoma, ovarian cancer, pancreatic cancer, pancreatic neuroendocrine tumors, pharyngeal cancer, pituitary tumor, prostate cancer, rectal cancer, renal cell cancer, retinoblastoma, skin cancer, small cell lung cancer, small intestine cancer, squamous neck cancer, testicular cancer, thymoma, thyroid cancer, uterine cancer, vaginal cancer, and vascular tumors.

In accordance with many embodiments, treatments involving administration of oncolytic virus and CAR-T cells can be combined with other therapies, including (but not limited to) surgery, immunotherapy, chemotherapy, radiation therapy, targeted therapy, hormone therapy, stem cell therapies, and blood transfusions. In some embodiments, an anti-cancer and/or chemotherapeutic agent is administered, including (but not limited to) alkylating agents, platinum agents, taxanes, vinca agents, anti-estrogen drugs, aromatase inhibitors, ovarian suppression agents, endocrine/hormonal agents, bisphosphonate therapy agents and targeted biological therapy agents. Medications include (but are not limited to) cyclophosphamide, fluorouracil (or 5-fluorouracil or 5-FU), methotrexate, thiotepa, carboplatin, cisplatin, taxanes, paclitaxel, protein-bound paclitaxel, docetaxel, vinorelbine, tamoxifen, raloxifene, toremifene, fulvestrant, gemcitabine, irinotecan, ixabepilone, temozolomide, topotecan, vincristine, vinblastine, eribulin, mitomycin, capecitabine, capecitabine, anastrozole, exemestane, letrozole, leuprolide, abarelix, buserelin, goserelin, megestrol acetate, risedronate, pamidronate, ibandronate, alendronate, zoledronate, tykerb, daunorubicin, doxorubicin, epirubicin, idarubicin, valrubicin mitoxantrone, bevacizumab, cetuximab, ipilimumab, ado-trastuzumab emtansine, afatinib, aldesleukin, alectinib, alemtuzumab, atezolizumab, avelumab, axitinib, belimumab, belinostat, bevacizumab, blinatumomab, bortezomib, bosutinib, brentuximab vedotin, brigatinib, cabozantinib, canakinumab, carfilzomib, ceritinib, cetuximab, cobimetinib, crizotinib, dabrafenib, daratumumab, dasatinib, denosumab, dinutuximab, durvalumab, elotuzumab, enasidenib, erlotinib, everolimus, gefitinib, ibritumomab tiuxetan, ibrutinib, idelalisib, imatinib, ipilimumab, ixazomib, lapatinib, lenvatinib, midostaurin, necitumumab, neratinib, nilotinib, niraparib, nivolumab, obinutuzumab, ofatumumab, olaparib, olaratumab, osimertinib, palbociclib, panitumumab, panobinostat, pembrolizumab, pertuzumab, ponatinib, ramucirumab, regorafenib, ribociclib, rituximab, romidepsin, rucaparib, ruxolitinib, siltuximab, sipuleucel-T, sonidegib, sorafenib, temsirolimus, tocilizumab, tofacitinib, tositumomab, trametinib, trastuzumab, vandetanib, vemurafenib, venetoclax, vismodegib, vorinostat, and ziv-aflibercept. In accordance with various embodiments, an individual may be treated, by a single medication or a combination of medications described herein. A common treatment combination is cyclophosphamide, methotrexate, and 5-fluorouracil (CMF).

Dosing and therapeutic regimes can be administered appropriate to the neoplasm to be treated, as understood by those skilled in the art. For example, 5-FU can be administered intravenously at dosages between 25 mg/m² and 1000 mg/m².

In some embodiments, medications are administered in a therapeutically effective amount as part of a course of treatment. As used in this context, to “treat” means to ameliorate at least one symptom of the disorder to be treated or to provide a beneficial physiological effect. For example, one such amelioration of a symptom could be reduction of tumor size and/or risk of relapse.

A therapeutically effective amount can be an amount sufficient to prevent reduce, ameliorate or eliminate the symptoms of cancer. In some embodiments, a therapeutically effective amount is an amount sufficient to reduce the growth and/or metastasis of a cancer.

Various embodiments are also directed to diagnostic scans performed after treatment of an individual to detect residual disease and/or recurrence of neoplastic growth. If a diagnostic scan indicates residual and/or recurrence of neoplastic growth, further treatments may be performed as described herein. If the neoplastic growth and/or individual is susceptible to recurrence, diagnostic scans can be performed frequently to monitor any potential relapse.

### Molecular Techniques for Polypeptide Expression

Various embodiments are directed toward the use of nucleic acid molecules encoding polypeptides or peptides (e.g., oncolytic virus transgene or CAR gene). Nucleic acid molecules may be sourced by methods known in the art, e.g., isolated from eukaryotic or prokaryotic cells, plasmids, vectors and the like.

In some embodiments, nucleic acid molecules may be used to express large quantities of polypeptides. If the nucleic acid molecules are derived from a non-human animal, the nucleic acid molecules may be used for humanization of the genes (e.g., modify nucleic acid sequence for codon optimization).

In some embodiments, an expression vector is utilized to express a gene product by incorporating the nucleic acid molecule encoding a gene product or a portion thereof (e.g., a fragment of gene product). In some embodiments, expression vectors are used to encode transgene antigens for use in an oncolytic virus or to encode a CAR product to be expressed within a T cell. In some embodiments, an expression vector includes regulatory sequences that govern transcription and translation that are operably linked to the gene produce sequence.

To express peptides or polypeptides of the disclosure, nucleic acids encoding the peptides or polypeptides are inserted into expression vectors such that the gene product sequence is operatively linked to transcriptional and translational regulatory sequences. The term “regulatory sequence” refers to nucleic acid sequences that are necessary to affect the expression of transgene sequences to which they are operably linked. Such regulatory sequences may include a promoter, a splice cassette, translation initiation codon, translation and insertion site for introducing an insert into the vector. The term “operably linked’ refers to a juxtaposition of a regulatory sequence with a transgene permitting them to function in their intended manner. A regulatory sequence “operably linked to a transgene sequence is ligated in such a way that expression of the transgene is achieved under conditions compatible with the control sequences. Examples of regulatory sequences permitting expression in eukaryotic host cells include (but are not limited to) the yeast regulator sequences AOX1 or GAL1 and the human regulatory sequences CMV-promoter, SV40-promoter, RSV-promoter, CMV-enhancer, SV40-enhancer and a globin intron. Regulatory elements may also include transcription termination signals, such as (for example) the SV40 poly-A site or the tk-poly-A site, typically operably linked downstream of the transgene.

Typically, expression vectors used in any of the host cells contain sequences for plasmid or virus maintenance and for cloning and expression of exogenous nucleotide sequences. Such sequences, collectively referred to as “flanking sequences” typically include one or more of the following operatively linked regulatory sequences: a promoter, one or more enhancer sequences, an origin of replication, a transcriptional termination sequence, a complete intron sequence containing a donor and acceptor splice site, a sequence encoding a leader sequence for polypeptide secretion, a ribosome binding site, a polyadenylation sequence, a polylinker region for inserting the nucleic acid encoding the polypeptide to be expressed, and a selectable marker element.

Numerous expression systems exist that include at least a part or all of the expression vectors discussed above. Prokaryote- and/or eukaryote-based systems can be employed for use with an embodiment to produce nucleic acid sequences, or their cognate polypeptides, proteins and peptides. Commercially and widely available systems include in but are not limited to bacterial, mammalian, yeast, and insect cell systems. Different host cells have characteristic and specific mechanisms for the post-translational processing and modification of proteins. Appropriate cell lines or host systems can be chosen to ensure the correct modification and processing of the foreign protein expressed. Those skilled in the art are able to express a vector to produce a nucleic acid sequence or its cognate polypeptide, protein, or peptide using an appropriate expression system.

Suitable methods for nucleic acid delivery to effect expression of compositions are anticipated to include virtually any method by which a nucleic acid (e.g., DNA, including viral and nonviral vectors) can be introduced into a cell, a tissue or an organism, as described herein or as would be known to one of ordinary skill in the art. Such methods include, but are not limited to, direct delivery of DNA such as by injection, including microinjection; by electroporation; by calcium phosphate precipitation; by using DEAE or polyethylene glycol; by direct sonic loading; by liposome mediated transfection; by microprojectile bombardment; and/or by agitation with silicon carbide fibers. Other methods include viral transduction, such as gene transfer by lentiviral or retroviral transduction.

In some embodiments, recombinant expression vector are contacted with host cells to induce transgene expression. An expression construct encoding a transgene can be transfected or transduced or infected into cells according to a variety of methods known in the art. Vector DNA can be introduced into prokaryotic or eukaryotic cells via conventional transformation or transfection techniques. One of skill in the art would understand the conditions under which to incubate host cells to maintain them and to permit replication of a vector. Also understood and known are techniques and conditions that would allow large-scale production of vectors, as well as production of the nucleic acids encoded by vectors and their cognate polypeptides, proteins, or peptides.

For stable transfection of mammalian cells, depending upon the expression vector and transfection technique used, typically only a fraction of cells (i.e., not 100%) will integrate the foreign DNA into their genome. In order to identify stably expressing cells within a population, a selectable marker (e.g., gene product inducing resistance to antibiotics or a fluorescent protein) is generally introduced into the host cells along with the gene of interest. Cells stably transfected with the introduced nucleic acid can be identified by drug selection (e.g., cells that have incorporated the selectable marker gene will survive, while the other cells die) or identifying and isolating fluorescent cells (e.g., via flow cytometry) or identifying and isolating cells with ectopic expression of a transgene product (e.g., via flow cytometry and tagging cells with fluorescent antibodies capable of detecting the transgene product), among other methods known in the arts.

### Virus Preparation

Several embodiments are directed towards design and preparation of oncolytic viruses. In various embodiments, viruses and/or viral vectors incorporate genetic information via RNA or DNA, as appropriate for the particular virus or vector. In some embodiments, the genetic polynucleotide is modified to achieve desired biological features that may be advantageous in a treatment. For example, in various embodiments, virus and viral vectors may be attenuated, rendered replication incompetent, and/or express one or more transgenes.

In various embodiments, an oncolytic virus or viral vector incorporates an expression cassette that is a nucleic acid sequence encoding one or more regulatory sequences operably linked to one or more transgenes. Accordingly, an oncolytic virus can contact a neoplastic cell and induce expression of a transgene therein. In some embodiments, an oncolytic virus induces expression of an ectopic antigen on the surface of a neoplastic cell.

Exemplary oncolytic viruses include (but not limited to) reovirus, Seneca Valley virus (SVV), vesicular stomatitis virus (VSV), Newcastle disease virus (NDV), herpes simplex virus (HSV), measles virus, adenovirus, and poxvirus (e.g., vaccinia virus).

In some embodiments, a reovirus is utilized as oncolytic virus to induce antigen expression in neoplastic cells. Representative examples of oncolytic reovirus are provided within the literature and include methods of development, propagation, and use (see, e.g., J. Gong, et al., World J Methodol. 2016; 6(1):25-42, the disclosure of which is incorporated herein by reference).

In some embodiments, a Seneca Valley virus is utilized as oncolytic virus to induce antigen expression in neoplastic cells. Representative examples of oncolytic SVV are provided within the literature and include methods of development, propagation, and use (see, e.g., M. J. Burke, Oncolytic Virother. 2016; 5:81-89, the disclosure of which is incorporated herein by reference).

In some embodiments, a vesicular stomatitis virus is utilized as oncolytic virus to induce antigen expression in neoplastic cells. Representative examples of oncolytic VSV are provided within the literature and include methods of development, propagation, and use (see, e.g., S. Bishnoi, et al., Viruses. 2018; 10(2):90, the disclosure of which is incorporated herein by reference).

In some embodiments, a Newcastle disease virus is utilized as oncolytic virus to induce antigen expression in neoplastic cells. Representative examples of oncolytic NDV are provided within the literature and include methods of development, propagation, and use (see, e.g., V. schirrmacher, et al., Biomedicines. 2019; 7(3):66, the disclosure of which is incorporated herein by reference).

In some embodiments, a herpes simplex virus is utilized as oncolytic virus to induce antigen expression in neoplastic cells. Representative examples of oncolytic HSV are provided within the literature and include methods of development, propagation, and use (see, e.g., D. Watanabe and F. Goshima, Adv Exp Med Biol. 2018; 1045:63-84, the disclosure of which is incorporated herein by reference). The Herpesviridae are a large family of DNA viruses that all share a common structure and are composed of relatively large double-stranded, linear DNA genomes encoding 100-200 genes encapsided within an icosahedral capsid which is enveloped in a lipid bilayer membrane. Although the oncolytic herpes virus can be derived from different types of HSV, particularly preferred are HSV1 and HSV2. The herpes virus may be genetically modified so as to restrict viral replication in tumors or reduce its cytotoxicity in non-dividing cells. For example, any viral gene involved in nucleic acid metabolism may be inactivated, such as thymidine kinase, ribonucleotide reductase (RR), and/or uracil-N-glycosylase.

In some embodiments, a measles virus is utilized as oncolytic virus to induce antigen expression in neoplastic cells. Representative examples of oncolytic measles virus are provided within the literature and include methods of development, propagation, and use (see, e.g., P. Msaouel, et al., Curr Cancer Drug Targets. 2018; 18(2):177-187, the disclosure of which is incorporated herein by reference).

In some embodiments, a measles virus is utilized as oncolytic virus to induce antigen expression in neoplastic cells. Representative examples of oncolytic measles virus are provided within the literature and include methods of development, propagation, and use (see, e.g., A. R. Shaw and M. Suzuki, Curr Opin Virol. 2016; 21:9-15, the disclosure of which is incorporated herein by reference). An advantageous strategy includes the replacement of viral promoters with tumor-selective promoters or modifications of the E1 adenoviral gene product(s) to inactivate its/their binding function with p53 or retinoblastoma (Rb) protein that are altered in tumor cells.

In some embodiments, a poxvirus is utilized as oncolytic virus to induce antigen expression in neoplastic cells. Representative examples of oncolytic poxvirus are provided within the literature and include methods of development, propagation, and use (see, e.g., L. E. Torres-Dominguez and G. McFadden, Expert Opin Biol Ther. 2019; 19(6):561-573, the disclosure of which is incorporated herein by reference).

In some embodiments, an oncolytic poxvirus is an oncolytic vaccinia virus. Vaccinia viruses are members of the poxvirus family characterized by a 200 kb double-stranded DNA genome that encodes numerous viral enzymes and factors that enable the virus to replicate independently from the host cell machinery. The majority of vaccinia virus particles are intracellular (IMV for intracellular mature virion) with a single lipid envelop and remains in the cytosol of infected cells until lysis. Another infectious form is a double enveloped particle (EEV for extracellular enveloped virion) that buds out from the infected cell without lysing it. Although any vaccinia virus strain can be utilized in the various embodiments described herein, Elstree, Wyeth, Copenhagen and Western Reserve strains have been shown to achieve desirable results.

In some embodiments, an oncolytic vaccinia virus is modified by altering for one or more viral genes. Genetic modifications encompass deletion, mutation and/or substitution of one or more nucleotide(s) (contiguous or not) within a viral gene or within regulatory elements. In some embodiments, an oncolytic vaccinia virus is modified by altering the thymidine kinase-encoding gene. The TK enzyme is involved in the synthesis of deoxyribonucleotides. TK is needed for viral replication in normal cells as these cells have generally low concentration of nucleotides whereas it is dispensable in dividing cells which contain high nucleotide concentration.

### CAR T Cell Engineering

A number of embodiments are directed towards design and genetic engineering of T cells to stably express a particular CAR. In various embodiments, T cells are genetically modified via viral vector transduction or site-directed mutagenesis such that the cells achieve desired biological features that may be advantageous in a treatment. For example, in various embodiments, T cell may have some endogenous receptors deleted or altered to prevent their activity and further incorporate expression cassettes to express a particular CAR.

Genetic engineering of T cells to express a particular CAR can produce antitumor effector cells that bypass tumor immune escape mechanisms that are due to abnormalities in protein antigen processing and presentation. Moreover, these transgenic receptors can be directed to recognize ectopic antigens that are provided by a complimentary oncolytic virus system. In various embodiments, an engineered T cell expresses one or more particular CAR products.

In some embodiments, an engineered CAR has one, two, three, four, or more components, and in some embodiments the one or more components facilitate targeting or binding of an ectopic antigen. In some embodiments, a CAR incorporates an antibody for binding the ectopic antigen, part or all of a cytoplasmic signaling domain, and/or part or all of one or more costimulatory molecules. In some embodiments, the antibody is a single chain variable fragment (scFv).

In some embodiments, a cytoplasmic signaling domain is employed as at least part of the chimeric receptor in order to produce stimulatory signals for T cell proliferation and effector function following engagement of the chimeric receptor with the target antigen. Examples of signaling domains include (but are not limited to) endodomains from costimulatory molecules such as CD28, CD27, 4-1BB (CD137), OX40 (CD134), ICOS, Myd88, and/or CD40. In some embodiments, costimulatory molecules are employed to enhance the activation, proliferation, and cytotoxicity of T cells produced by the CAR after antigen engagement.

In some embodiments, T cells are further genetically modified to enhance their function. Examples of genetic modification include (but are not limited to) transgenic expression of cytokines (e.g. IL2, IL7, IL15), silencing of negative regulators (for example SHP-1, FAS, PD-L1), chemokine receptors (e.g. CXCR2, CCR2b), dominant negative receptors (e.g. dominant negative TGFBRII), and/or signal converters that convert a negative into a positive signal (e.g. IL4/IL2 chimeric cytokine receptor, IL4/IL7 chimeric cytokine receptor, or TGFBRII/TLR chimeric receptor).

In various embodiments, T cells are “transfected” or “transformed,” which refers to a process by which exogenous nucleic acid is transferred or introduced into the host cell. A transformed T cell includes the primary subject cell and its progeny. As used herein, the terms “engineered” and “recombinant” T cells are intended to refer to a cell into which an exogenous nucleic acid sequence, such as (for example) an expression cassette expressing a particular CAR. Therefore, engineered T cells are distinguishable from naturally occurring T cells which do not contain a recombinantly introduced nucleic acid.

T cells can be genetically engineered by any appropriate technique that can stably introduce one or more expression cassettes. In some embodiments, an expression cassette is integrated into the T cell's genome or maintained extrachromosomally. In some embodiments, a viral vector (e.g., lentiviral vector) is utilized to introduce an expression cassette. Accordingly, a viral vector is incorporating the expression cassette developed and propagated, and then transduced into T cells at an appropriate multiplicity of infection (MOI), which may ensure robust transgene expression but mitigate harmful side effects of vector integration. In some embodiments, site-directed mutagenesis (e.g., CRISPR) mutagenesis is utilized to introduce an expression cassette. Accordingly, in various embodiments utilizing a CRISPR technique, an expression cassette utilizes Cas9 enzymes (or similar) and guide RNAs to nick and/or break DNA at a desired genomic locations such that a donor expression cassette is integrated at the desired site. In some embodiments, T cells that successfully integrate and/or stably extrachromosomally maintain the expression cassette is selected, which can be done by identifying and purifying cells expression the transgene product and/or utilizing a co-expressed selectable marker (e.g., fluorescent protein, puromycin, hygromycin, etc.).

### Kits

Numerous embodiments are directed towards kits for treating a neoplasm. In some embodiments, a kit includes an oncolytic virus that expresses an ectopic transgene. In some embodiments, a kit includes an expression vector for genetically engineering T cells to express a complementary CAR capable of recognizing the ectopic antigen. In some embodiments, a kit includes the tools and/or reagents for performing the various methods as described herein. In some embodiments, a kit includes the appropriate buffers and/or solutions to store and preserve oncolytic viruses, expression vectors, tools and/or reagents.

### EXEMPLARY EMBODIMENTS

The embodiments described will be better understood with the several examples provided within. Many exemplary results of utilizing oncolytic virus and CAR-T cells for treatments of neoplasms are described. Description of genetic manipulation of T cells to induce CAR expression and transduction of neoplastic cells with an oncolytic virus to induce ectopic expression are also described.

### Example 1: Delivery of CAR Targets with an Oncolytic Virus

Chimeric antigen receptor (CAR) T-cell therapy has emerged as a promising curative cancer immunotherapy, with CD19-targeting CAR T-cells having achieved durable remissions in the setting of therapy-resistant B-cell malignancies. Translating these successes to solid tumors is arguably the most pressing challenge facing the field.

Solid tumors present several challenges for effective CAR T-cell therapy which have limited the success of efforts to date. For one, solid tumors are often populated with myeloid derived suppressor cells and tumor associated macrophages which contribute to an immunosuppressive, anti-inflammatory microenvironment that can inhibit T-cell function. Second, while lineage restricted antigens such as CD19 are uniformly expressed and B-cell aplasia is a clinically manageable phenotype, identifying solid tumor CAR targets which are both uniformly and selectively expressed on malignant cells to prevent “off-tumor, on-target” toxicities has shown to be problematic. Moreover, CAR T-cell recognition of target cells is highly dependent on antigen-density, suggesting that even solid tumors which do selectively express tumor-associated targets may still be unresponsive to therapy should they express low levels of antigen or downregulate levels of antigen.

To date, efforts to overcome these challenges have primarily focused on CAR T-cell potency and persistence. Combination therapies involving radiation, oncolytic viruses, and checkpoint blockade have been proposed to circumvent the immunosuppressive effects of the tumor microenvironment, reverse T-cell exhaustion, and prevent antigen-escape. Genetic engineering has also been used to disrupt negative regulators of T-cell activation or “armor” T-cells with activation-sustaining cytokines.

But despite novel methods of improving CAR T-cell function, little effort has been dedicated to engineering tumors for effective adoptive cell therapy, and the lack of targetable surface antigens that are selectively expressed on malignant cells remains a major unaddressed challenge. This limitation also necessitates that new CARs be designed for each new proposed antigen, and to this end CAR T-cells targeting putative solid tumor antigens including GD2, mesothelin, BCMA, PSMA, EGFRvIII, MUC1, and NY-ESO-1 are all under active investigation.

To overcome the challenge of relying on endogenous solid tumor antigens, herein is described a method of selectively delivering an ectopic CAR target antigen to malignant cells using an oncolytic virus to enable a universal approach to solid tumor CAR T-cell therapy. Oncolytic viruses are ideal partners in this regard as they (i) can selectively infect and/or replicate in malignant cells and thus will not deliver the CAR target to healthy tissues; (ii) have been shown to reprogram the immunosuppressive microenvironment of solid tumors into one that is more conducive to T-cell activity; and (iii) have the capacity to selectively lyse infected cells and recruit the endogenous anti-viral immune response.

In this proof-of-concept, a thymidine kinase deleted (TK-) oncolytic vaccinia virus (VV) was designed to selectively induce CD19 expression on malignant cells and thereafter treat the tumors with CD19-targeted CAR T-cells. This method represents an important conceptual advance in a tumor-centric approach to CAR T-cell therapy and could potentially enable a universal approach to solid tumor CAR T-cell therapy that is agnostic to a tumor's native surface expression profile.

mCD19 CAR T-Cells Exhibit Activity Against mCD19 Positive Melanoma Cells

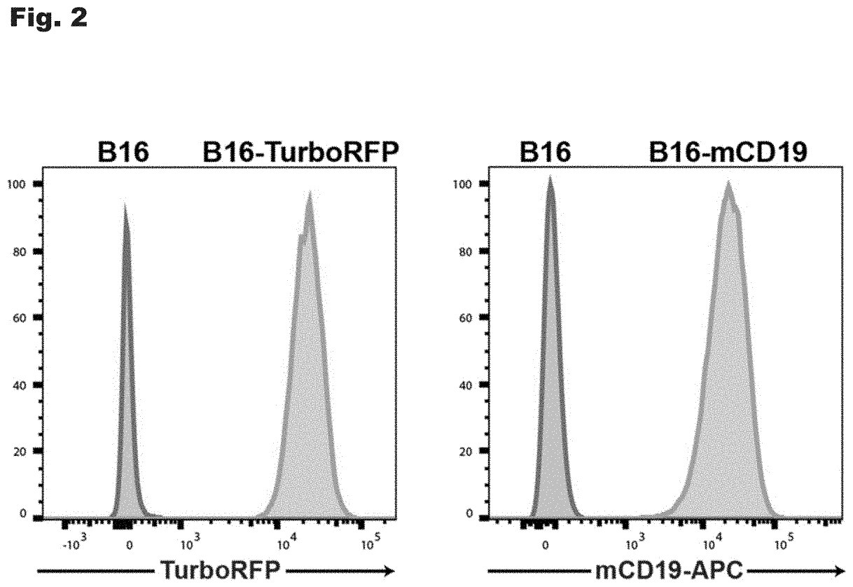

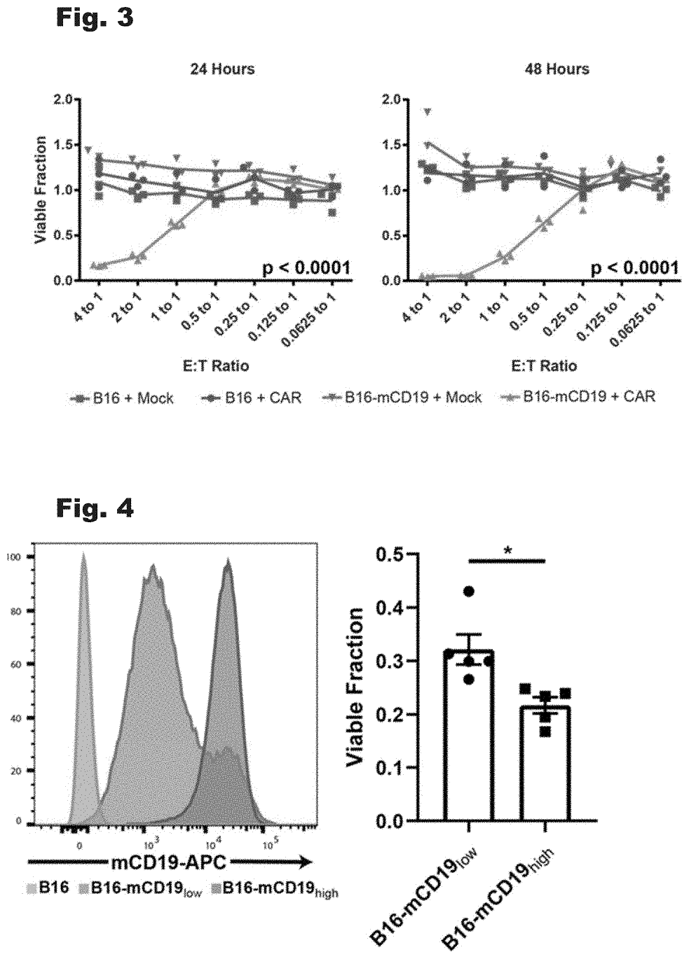

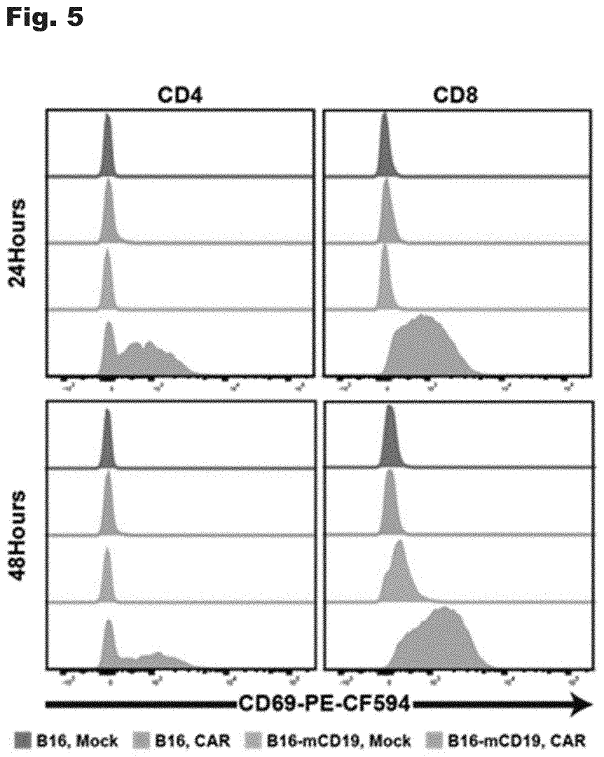

To characterize the activity of primary murine CD19 (mCD19) CAR T-cells against a solid tumor uniformly expressing mCD19, first a mCD19 and a TurboRFP/Renilla luciferase (TR) fusion protein was stably expressed in a B16 mouse melanoma cell line (FIG. 2). Second generation murine mCD19 CAR T-cells containing CD3 (and CD28 costimulatory domains exhibited potent cytotoxicity against the engineered B16-TR-mCD19 cell line in co-culture assays at effector to tumor (E:T) ratios of 0.5 and higher as measured by bioluminescence (p<0.0001, n=3 for each condition) (FIG. 3). mCD19-negative B16 viability was not affected even at an E:T ratio of 4:1. Mock T-cells, which were similarly activated in culture but untransduced, also lacked activity against either mCD19 positive or negative B16 cells. CD19 CAR T-cell toxicity was also dependent on antigen-density, with a B16-mCD19_{low} cell line exhibiting increased resistance to therapy compared to a B16-mCD19_{high} cell line (p=0.0116, n=5 for each condition) (FIG. 4). Antigen-specific T-cell cytotoxicity was confirmed by upregulation of the early T-cell activation marker CD69 on both CD4 and CD8 T-cells in only the properly matched B16-mCD19+mCD19 CAR T-cell condition (FIG. 5).

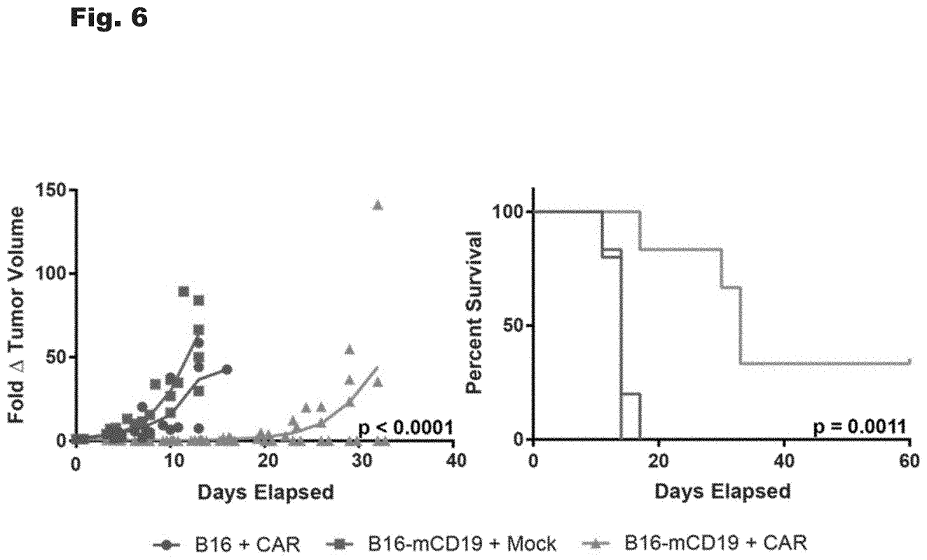

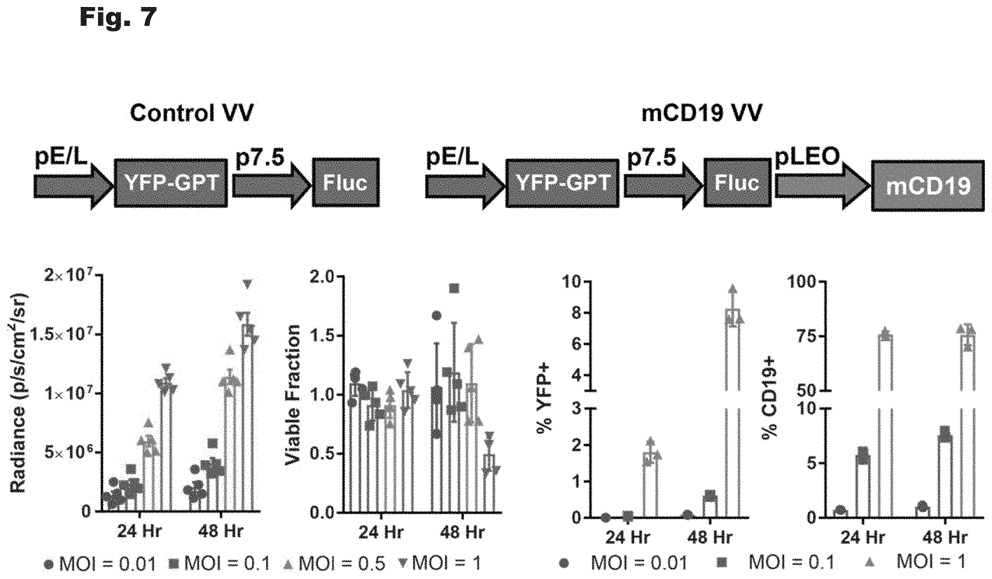

To assess the solid tumor activity of mCD19 CAR T-cells in vivo, orthotopic, syngeneic models of B16 and B16-mCD19 melanomas were established and treated with either intratumoral mCD19 CAR T-cells or mock T-cells following a sub-lethal lymphodepletive regimen of 5 Gy total body irradiation (TBI). Similar to the in vitro findings, the antigen-matched therapy group exhibited delayed tumor growth in all mice and completely eliminated the tumors in 33% of the mice (p<0.0001, n=5 B16+CAR, n=6 B16-mCD19+Mock, n=6 B16-mCD19+CAR) (FIG. 6). Together, the data suggest that mCD19 CAR T-cells can exhibit potent activity in solid tumors made to express ectopic mCD19.

Recombinant Vaccinia Virus can Deliver mCD19 to Malignant Cells

In order to selectively deliver an ectopic surface protein to malignant cells, recombinant VVs were generated with transgenes inserted into the viral TK locus. TK-disrupted VV is reliant on cellular TK for replication and can selectively propagate in tumor cells given their higher rates of nucleotide turnover. A control oncolytic VV (control VV) to express firefly luciferase (FLuc) yellow fluorescent protein (YFP) oncolytic VV (control VV) expressing and an oncolytic VV to express mCD19 (mCD19 VV) were designed (FIG. 7). Efficient VV replication in B16 cells was confirmed by time- and dose-dependent expression of Fluc, YFP, and mCD19 (FIG. 7) with up to 75% of cells expressing mCD19 at 48 hours of culture with virus at a multiplicity of infection (MOI) of 1. Despite detectable transgene expression, the oncolytic virus did not induce significant cell death at an MOI or 0.01 or 0.1, highlighting the therapeutic limits of oncolytic virotherapy as a single agent without CAR T-cell activity (FIG. 7).

Infection with mCD19 VV Enables Antigen-Specific mCD19 CAR T-Cell Activity

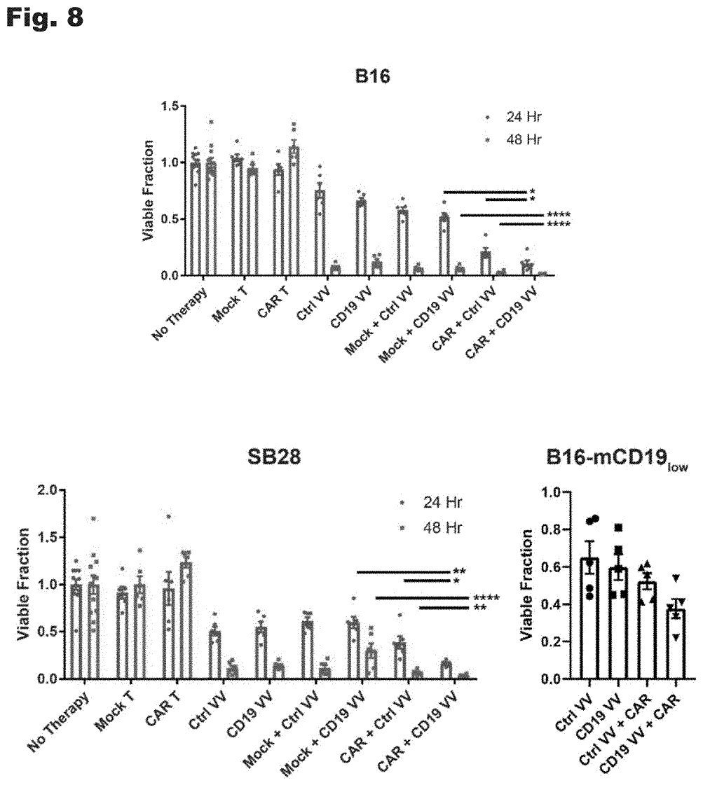

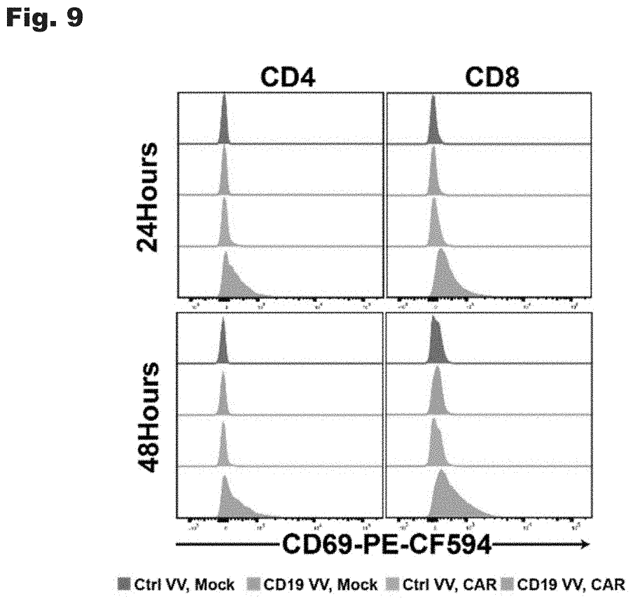

It was next aimed to show that oncolytic virus-driven expression of ectopic CAR targets could enable selective clearance by antigen-matched CAR T-cells (FIG. 1). Cultured cells were infected with control or mCD19 VV for 48 hours prior to addition of either mCD19 CAR T-cells or mock T-cells. Toxicity profile of each type of VV or T-cell as a monotherapy was also evaluated. In B16 co-cultures, the combination of mCD19 VV together with mCD19 CAR T-cells exhibited the highest toxicity at 24 and 48 hours following addition of T-cells (FIG. 8). This synergy was also replicated in the SB28 murine glioma cell line, suggesting the generalizability of the approach across tumor types. VV-mediated mCD19 delivery also augmented CAR T-cell activity against the B16-mCD19_{low} cell line, highlighting potential uses of this approach to “boost” levels of tumor-associated surface antigen prior to therapy or as a method of overcoming antigen-low resistance. Enhanced cytotoxicity was mirrored by selective upregulation of CD69 on both CD4 and CD8 T-cells only in the antigen-matched combination (FIG. 9).

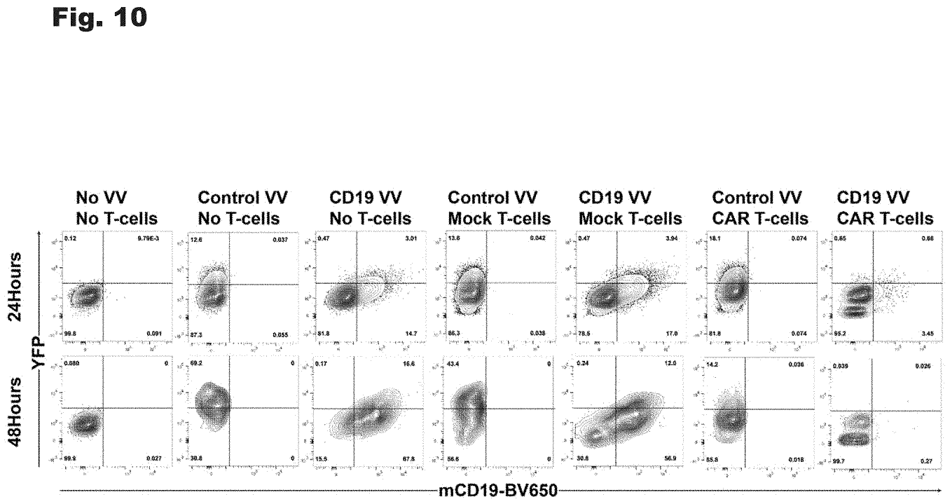

Flow cytometry of B16 cells remaining 24 or 48 hours after addition of T-cells further confirmed the mechanism of cell death (FIG. 10). In the absence of T-cells, both control and mCD19 VV expectedly induced expression of mCD19 and/or YFP. Addition of mock T-cells did not affect these profiles, and similarly YFP expression from control VV still persisted following addition of CAR T-cells. Notably, CAR T-cells eliminated all mCD19+ and YFP+ cells by 48 hours after co-culture with mCD19 VV-infected cells.

Tumor-Selective Delivery of mCD19 Potentiates mCD19 CAR T-Cell Killing In Vivo

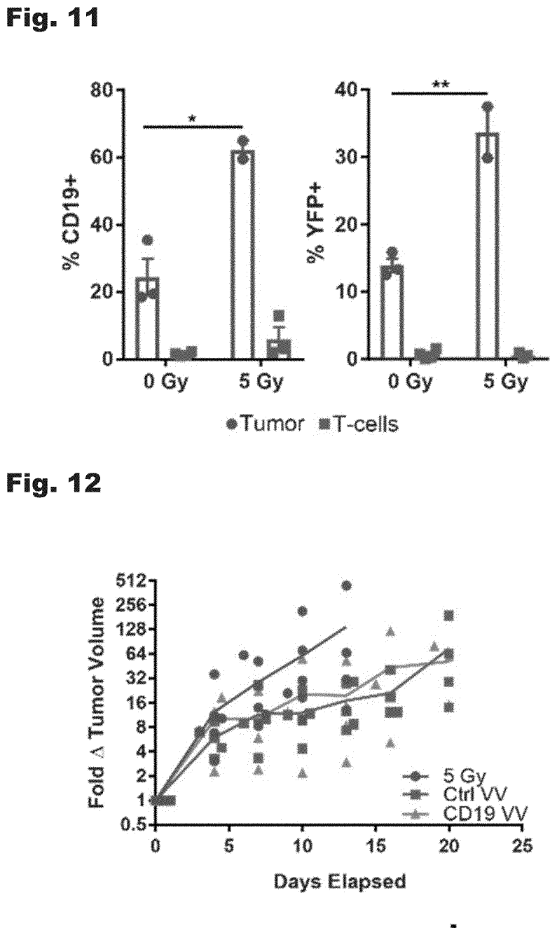

To assess the ability to force tumor expression of mCD19 in vivo, three doses of 10⁸ plaque-forming units (PFU) mCD19 VV were injected into orthotopic B16-TR tumors. Doses were separated by 24 hours, and YFP and mCD19 expression on TR+ cells from resected tumors were measured one day following the final viral dose. Without any lymphodepletive regimen, on average 24% mCD19+ and 14% YFP+ cells were observed within the TR+ population (FIG. 11). Rates of intratumoral T-cell infection were markedly lower, highlighting the tumor-selectivity of TK-deleted VV and minimizing concerns of potential CAR T-cell fratricide.

Oncolytic virotherapy as a single agent benefits from both direct lysis of infected tumor cells as well as recruitment of the endogenous anti-viral response. While beneficial, this endogenous immune response can also impair viral propagation. Since patients receiving CAR T-cell therapy undergo lymphodepletive regimens of chemotherapy or radiation to enable expansion of the adoptively transferred cells, it was hypothesized that this lymphopenia may also serve an added benefit of allowing enhanced oncolytic virus spread throughout a tumor. Consistent with this hypothesis, it was observed that lymphodepletion with 5 Gy TBI (n=2) markedly increased the proportion of mCD19+ (62%, p=0.0144) and YFP+ (34%, p=0.008) cells in the TR+ population compared to the no TBI group (n=3). In this same model, no significant differences in in vivo anti-tumor efficacy was observed between the control (n=5) and mCD19 (n=4) VV and both achieved modest delays (day 11 tumor volume Control VV vs. TBI only p=0.004 and mCD19 VV vs. TBI only p=0.0246) in tumor growth relative to the TBI only group (n=6) (FIG. 12).

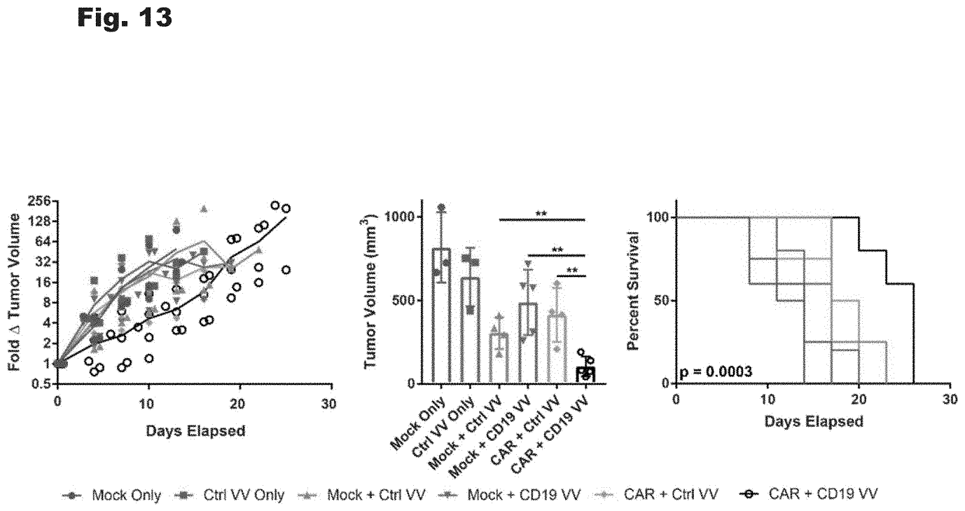

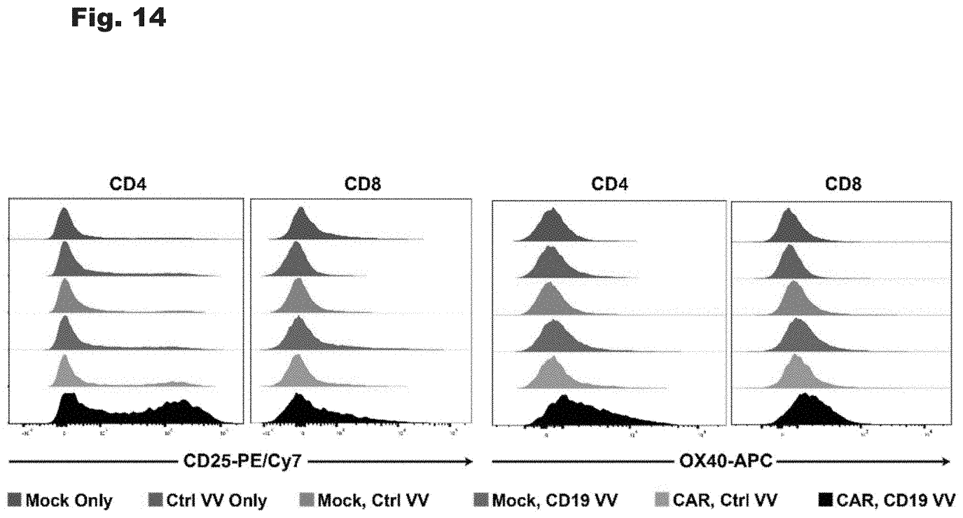

Encouraged by the effective in vivo delivery of mCD19 and motivated by the limited anti-tumor efficacy of the oncolytic virus as a monotherapy, it was examined whether mCD19 CAR T-cells could be redirected to engage with B16 cells infected with mCD19 VV in vivo. VV was again administered as a three dose regimen, and mice were treated with 5 Gy TBI and intratumoral injection of T-cells on the same day as the first virus injection. All combinations of VV (control or mCD19) and T-cells (mock or CAR) were evaluated. Consistent with the in vitro results, the antigen-matched combination achieved a significantly (CAR+control VV (n=4) vs. CAR+CD19 VV (n=5) day 11 tumor volume p=0.0051) higher delay in tumor progression observable as early as 4 days following initiation of therapy (FIG. 13). This translated to a ˜50% increase in median survival following therapy from 17 days (mock+CD19 VV) and 18.5 days (CAR+Ctrl VV) to 26 days (CAR+CD19 VV) (FIG. 13). Analysis of intratumoral T-cells two days following the final dose of VV (6 days after adoptive transfer) revealed a substantial upregulation of activation markers CD25 and OX40 on both CD4 and CD8 T-cells in the antigen-matched combination therapy relative to the other treatment groups, further confirming antigen-specific T-cell clearance of the melanoma (FIG. 14).

### Conclusions of Example 1

Motivated by the lack of solid tumor surface antigens that can be efficiently targeted with CAR T-cells, within this example is described a method of tumor-selective delivery of CAR targets using an oncolytic virus to enable a universal approach to adoptive cell therapy. Using mCD19 as a model antigen, TK-vaccinia virus can selectively deliver a surface antigen within in vitro and in vivo settings, which can then be targeted by cognate CAR T-cells with high specificity. It was further demonstrated that the approach is generalizable to multiple tumor types and can be effective for both tumors that do not express the ectopic target or express it at low levels.

Many features of the approach are modular. While a proof of concept was demonstrate using TK-deleted vaccinia virus given its large packaging capacity, ease of genetic manipulation, and clinical translatability, alternate oncolytic viruses, in particular RNA viruses, with higher replicative rates but smaller packaging limits could also be adapted as antigen-delivery vehicles. In addition, though the TK-deleted vaccinia virus exhibited only minimal infection of non-tumor cells (T-cells), other more tumor selective stains such as the double deleted strain of vaccinia (J. A. McCart, et al., Cancer Research 61, 8751 (2001), the disclosure of which is incorporated herein by reference) could further mitigate off-target replication. Although approximately 60% of tumor cells were successfully infected in vivo in lymphodepleted hosts, there is also potential for using multiple oncolytic viruses with alternate mechanisms of tumor selectivity in order to increase tumor coverage. In the same vein, delivery of multiple CAR targets could also be an effective strategy in combination with emerging dual-targeting CAR T-cells. There is also evidence that CAR T-cells can induce epitope spreading and prime the endogenous anti-tumor response against otherwise non-immunogenic tumor neoantigens. Future work will investigate whether the combinatorial strategy described here can recapitulate this effect and trigger anti-tumor responses at non-treated tumor foci. Effective epitope spreading can mitigate the need for the oncolytic virus to infect every malignant cell.

Lastly, while CD19 was used in the proof-of-concept given that the CD19 CAR is the most clinically advanced, other surface antigens and cognate CARs can be employed, which can help avoid unnecessary B-cell aplasia. Examples include viral surface proteins not present in mammalian hosts or mammalian proteins that are only expressed embryonically such as placental alkaline phosphatase. Regardless, it is envisioned that this approach to be first used to deliver existing solid tumor CAR targets to augment antigen levels prior to therapy, or in the setting of antigen-low acquired resistance. In this way, tumor engineering will emerge can be utilized as a complimentary approach to immune engineering in adoptive cell therapy.

### Methods of Example 1

Cell lines: B16 murine melanoma was obtained from ATCC (Manassas, Va.) and SB28 murine glioma was obtained as a gift from Dr. Hideho Okada. Cells were cultured in DMEM supplemented with 10% fetal bovine serum (FBS) and 1% antibacterial/antimycotic solution (ThermoFisher, Waltham, Mass.) and maintained in a humidified, 5% CO₂ incubator at 37° C. B16-TurboRFP/RLuc8 and SB28-TurboRFP/RLuc8 cell lines were generated by lentiviral transduction followed by three rounds of sorting for the highest 2.5% of TurboRFP expressers. B16-mCD19 and B16-mCD19-TurboRFP/RLuc8 cell lines were generated by transfection with Lipofectamine 3000 (ThermoFisher) and three rounds of sorting for the highest 2.5% of mCD19 expressers.

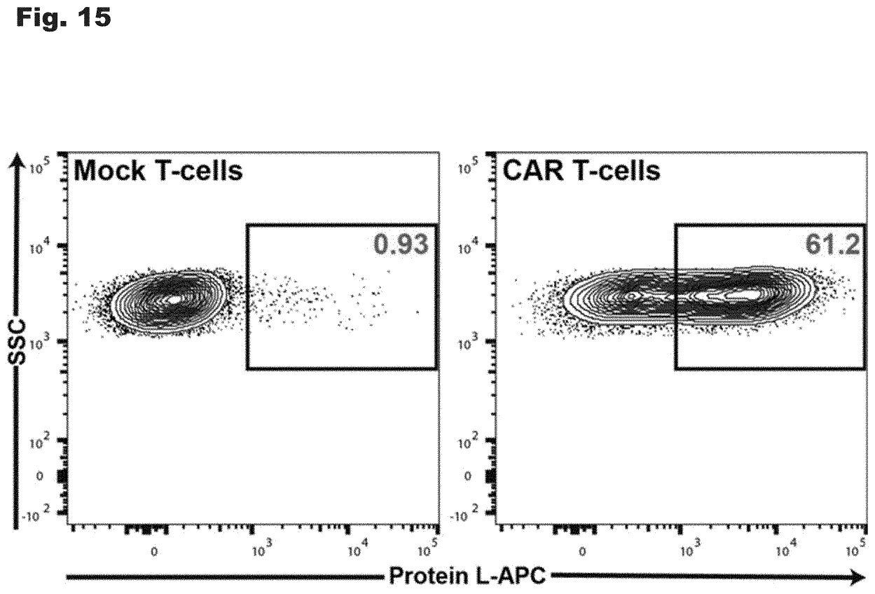

Murine T cellisolation and CAR T cell generation: Primary murine T-cells were isolated from spleens of healthy 6-8 week old C57B/6J mice using the EasySep™ Mouse T Cell Isolation Kit (STEMCELL Technologies, Vancouver, Canada) following manufacturer instructions and activated for 24 hours in RPMI supplemented with 10% fetal bovine serum (FBS), 1% antibacterial/antimycotic solution, 50 μM 2-mercaptoethanol (ThermoFisher), 10 ng/mL of each IL-2 and IL-7 (Peptrotech, Rocky Hill, N.J.), and CD3/CD28 Mouse T-cell Activation Dynabeads™ (ThermoFisher) at a bead to cell ratio of 1:1. Retrovirus encoding the second-generation mCD19 CAR with CD3 and CD28 co-stimulatory domains was centrifuged for 3 hours at 3200 RPM on non-adherent 6-well plates which had been coated overnight at 4° C. with 24 μg RetroNectin (Takara Bio, Kusatsu, Shiga Prefecture, Japan) per well. The mCD19 CAR retrovirus producer cell line was obtained as a gift from Dr. Crystal Mackall. Viral supernatant was then removed and 1×10⁶ T-cells were added in 4 mL of media per well. Mock T-cells were maintained in identical activation conditions but were not transduced with the CAR vector. After 48 hours of transduction, CD3/CD28 activation beads were removed and both mock and CAR T-cells were transferred to fresh medium supplemented with 10 ng/mL of each IL-2 and IL-7. Transduction efficiency (generally 50-60%) was measured by Protein L staining (FIG. 15) (ThermoFisher) 24 hours thereafter and cells were used on the same day. Administered doses of CAR T-cells were normalized based on transduction efficiency and an equivalent number of total T-cells were used in mock T-cell conditions.

Recombinant vaccinia virus generation: mCD19 under control of the pLEO (synthetic late-early optimized promoter) was cloned into the previously described pSEM-1 vector (A. C. Filley and M. Dey, Frontiers in Oncology 7, 106 (2017), the disclosure of which is incorporated herein by reference) that expresses Fluc and a YFP/guanine phosphoribosyltransferase fusion protein (YFP/GPT) under control of pE/L (early/late promoter) and p7.5 (vaccinia 7.5-kDa early promoter), respectively. U2OS cells were infected with the VV Copenhagen strain at an MOI of 0.01 for 2 h and then transfected with plasmid pE/L-YFP/GPT-p7.5-Fluc-pLEO-mCD19 using Lipofectamine 2000 (Thermo Fisher). The cells were incubated at 37° C. for 4 h, the medium was replaced, and cells were cultured for an additional 48 h. Viruses were released from the cells by three freeze-thaw cycles at −80° C. The harvested viruses were used to infect a monolayer of U2OS cells. Virus inoculum was removed from the cells after 1.5 h, and complete DMEM containing 10% FBS and 1.5% carboxymethylcellulose (CMC) was added. YFP-positive virus plaques were plaque purified for six rounds of selection using U2OS cells. The plaque-purified virus was subject to 36% sucrose cushion purification and resuspended in Tris-HCl (pH 9.0).

Vaccinia propagation and titer determination: Vaccinia viruses were expanded in HeLa cells (ATCC) by infecting ˜95% confluent flasks at a multiplicity of infection of 0.1 and harvesting the cells by mechanical scraping 48 hours thereafter once sufficient cytopathic effect was observed. Harvested cells were pelleted, resuspended in 40 mL of 1 mM Tris pH 9.0, and subjected to three freeze-thaw cycles to lyse the cells. Resulting cell debris was removed by centrifugation and the cleared lysate was subjected to sucrose cushion purification with a 36% sucrose cushion and centrifugation at 11,500 rpm for 90 minutes at 4° C. Resulting viral pellets were resuspended in 1 mM Tris and stored at −80° C.

Viruses were tittered by plaque assay using U2OS cells (ATCC). Briefly, 3.33×10⁶ cells were plated overnight in each well of a 12-well plate and infected with purified virus serially diluted in serum free DMEM at 37° C. After 2 hours, the virus was removed and replaced with 1 mL of 3% carboxymethylcellulose (Sigma-Aldrich, St. Louis, Mo.) mixed 1:1 with 2×DMEM containing 20% FBS. After three days of incubation, the overlay was aspirated, cells washed once with PBS, and stained with 1 mL of a 0.1% crystal violet solution (Sigma-Aldrich) for 10 minutes at room temperature. Cells were washed once with distilled water and plaques were counted to determine viral titer.

Cytotoxicity assays: To evaluate the cytotoxicity of CAR or mock T-cells as single agents, 20,000 B16-TurboRFP/RLuc8 or B16-TurboRFP/RLuc8-mCD19 cells were plated in 100 μL of DMEM in black 96-well plates. The following day, T-cells were added at specified effector to tumor ratios based on initial number of plated tumor cells, and viable fraction of tumors cells was measured 24 and 48 hours thereafter by comparing fractional bioluminescence signal from RLuc8 between treated and untreated wells. Imaging of RLuc8 was performed on an IVIS-50 system (PerkinElmer, Waltham, Mass.) immediately after washing cells once with PBS and addition of 200 μL of 1 μg/mL coelenterazine (NanoLight Technologies, Pinetop, Ariz.) to each well.

In combination studies with both vaccinia virus and T-cells, B16-TurboRFP/RLuc8 or SB28-TurboRFP/RLuc cells were instead plated at 10,000 cells/well in 100 μL DMEM. The following day, the media was removed and replaced with 150 μL DMEM containing either control of mCD19 VV at an MOI of 0.2 (B16), 0.05 (SB28), or 0.1 (B16-mCD19_{low}). After 48 hours of infection, T-cells were added at an effector to tumor ratio of 4:1 (B16 and SB28) or 1:1 (B16-mCD19_{low}) relative to initial number of plated cells to account for cell doubling, and viability was assayed as previously described.

Tumor harvesting: Resected B16 tumors were digested in 4 mL Hank's Balanced Salt Solution (HBSS) containing 10 μg/mL DNase I (Sigma-Aldrich) and 100 μg/mL Liberase TL (Roche, Basel, Switzerland) for 60 min at 37° C. The solution was then diluted with PBS, filtered through a 70 μm filter, and spun at 1,200×rpm for 5 min. Cells were resuspended in 1 mL ACK lysis buffer on ice for 5 min, washed once with PBS, and finally resuspended in PBS for staining.

Flow cytometry: Single cell suspensions were first stained with LIVE/DEAD™ Fixable Aqua Dead Cell Stain Kit (ThermoFisher) following manufacturer instructions and then resuspended for 10 minutes in 100 μL FACS buffer (PBS+2% FBS) with 1 μg per 10⁶ TruStain FcX™ Antibody (Biolegend, San Diego, Calif.) to block Fc receptors. When measuring rates of in vivo infection, both live and dead tumor cells were included in the analysis to better estimate rates of transduction. Flow antibodies were used at a concentration of 0.2 μg per 10⁶ cells in 100 μL volume when concentration was provided and following manufacturer instructions if concentration was unspecified. The following antibodies were used for staining: BUV395 Hamster Anti-Mouse CD3e Clone 145-2C11 (BD Biosciences, San Jose, Calif. CAT #563565), PerCP/Cyanine5.5 Rat Anti-Mouse CD4 Clone RM4-5 (Biolegend CAT #100540), Brilliant Violet 650™ Rat Anti-Mouse CD19 Clone 6D5 (Biolegend CAT #115541), PE Rat Anti-Mouse CD19 Clone 6D5 (Biolegend CAT #115508), APC Rat Anti-Mouse CD19 Clone 6D5 (Biolegend CAT #115512), PE-CF594 Hamster Anti-Mouse CD69 Clone H1.2F3 (BD Biosciences CAT #562455), APC Rat Anti-Mouse OX40 Clone OX-86 (Biolegend CAT #119414), PE/Cy7 Rat Anti-Mouse CD25 Clone PC61 (Biolegend CAT #102016). Following staining for 20 minutes on ice, cells were washed once with PBS, fixed in 4% paraformaldehyde for 20 minutes on ice, washed twice, and resuspended in FACS buffer for analysis on a LSRII analyzer (Becton Dickinson, Franklin Lakes, N.J.).

In vivo therapy studies: All animal experiments were performed under a protocol approved by the Stanford University Administrative Panels on Laboratory Animal Care (APLAC) and conducted in accordance with ethical guidelines prescribed therein. 6-8 week old female C57Bl/6 were implanted subcutaneously on the right shoulder with between 5×10⁵ and 10⁶ B16 or B16-mCD19 cells in 50-75 μL PBS. Tumors were allowed to grow for on average 7 days and therapy was initiated once tumors reached an average volume of 25-50 mm³. Mice were irradiated with 5 Gy, administered 10⁷ intratumoral CAR or mock T-cells in 50 μL PBS, and injected intratumorally with 10⁸ vaccinia virus in 30 μL PBS on the first day of therapy. Vaccinia was administered again on days 3 and 5 for a total of three injections. Mice receiving T-cells also received intraperitoneal injections of 10 μg recombinant human IL-2 (Peprotech) twice daily for the first three days of the therapy. Tumor size was measured by caliper every third day and mice were sacrificed if any dimension exceeded 15 mm or ulceration exceeded 0.5 cm².

Statistical analysis: Statistical analysis was performed using ordinary one-way ANOVA or two-tailed unpaired t tests. Statistical differences in survival curves were determined using Mantel-Cox tests. All statistical analysis was performed in GraphPad Prism Version 8.0.2.

### DOCTRINE OF EQUIVALENTS

While the above description contains many specific embodiments of the invention, these should not be construed as limitations on the scope of the invention, but rather as an example of one embodiment thereof. Accordingly, the scope of the invention should be determined not by the embodiments illustrated, but by the appended claims and their equivalents.

What is claimed is:

1. A method of treatment of an individual having a neoplasm, comprising:
administering to an individual an oncolytic virus capable of selectively expressing an ectopic antigen on the surface of neoplastic cells;
administering to the individual genetically engineered T cells that express a complementary chimeric antigen receptor that recognizes the ectopic antigen.

2. The method of claim 1, wherein the T cells are autologous.

3. The method as in claim 2, further comprising
harvesting T cells from the individual;
genetically engineering the T cells to express the complimentary chimeric antigen receptor that recognizes the ectopic antigen.

4. The method of claim 3, wherein the T cells are genetically engineered by viral vector transduction or site-directed mutagenesis.

5. The method of claim 3, wherein the T cells are further genetically modified to enhance T cell function.

6. The method as in claim 1, wherein the ectopic antigen is an exogenous antigen.

7. The method as in claim 1, wherein the ectopic antigen is an endogenous antigen that is expressed by the neoplastic cells.

8. The method as in claim 7, wherein the ectopic antigen is GD2, mesothelin, BCMA, PSMA, EGFRvIII, MUC1, or NY-ESO-1.

9. The method as in claim 1, wherein the antigen is CD19.

10. The method as in claim 1, wherein the oncolytic virus is derived from reovirus, Seneca Valley virus (SVV), vesicular stomatitis virus (VSV), Newcastle disease virus (NDV), herpes simplex virus (HSV), measles virus, adenovirus, or poxvirus.

11. The method as in claim 1, wherein the oncolytic virus is derived from vaccinia virus.

12. The method as in claim 11, wherein expression of Thymidine Kinase by the vaccinia is disrupted.

13. The method as in claim 1, wherein the oncolytic virus is parenterally administered.

14. The method as in claim 1, wherein the oncolytic virus is intratumorally administered.

15. The method as in claim 1, wherein the engineered T cells are administered via intravenous, intra-arterial, or intralymphatic delivery.

16. The method as in claim 1, wherein the treatment is further combined with surgery, immunotherapy, chemotherapy, radiation therapy, targeted therapy, hormone therapy, stem cell therapies, or blood transfusions.

17. The method as in claim 16 further comprising administering to the individual a chemotherapeutic agent.

18. The method as in claim 1, wherein the administration of the oncolytic virus and the administration of the genetically engineered T cells is performed as part of an adjuvant or a neoadjuvant treatment.

19. The method as in claim 1, wherein the neoplasm is anal cancer, astrocytomas, basal cell carcinoma, bile duct cancer, bladder cancer, breast cancer, breast adenocarcinoma (BRCA), cervical cancer, chronic myeloproliferative neoplasms, colorectal cancer, endometrial cancer, ependymoma, esophageal cancer, diffuse large B-cell lymphoma (DLBCL), esthesioneuroblastoma, Ewing sarcoma, fallopian tube cancer, gallbladder cancer, gastric cancer, gastrointestinal carcinoid tumor, hepatocellular cancer, hypopharyngeal cancer, Kaposi sarcoma, Kidney cancer, Langerhans cell histiocytosis, laryngeal cancer, liver cancer, lung cancer, melanoma, Merkel cell cancer, mesothelioma, mouth cancer, neuroblastoma, non-small cell lung cancer, osteosarcoma, ovarian cancer, pancreatic cancer, pancreatic neuroendocrine tumors, pharyngeal cancer, pituitary tumor, prostate cancer, rectal cancer, renal cell cancer, retinoblastoma, skin cancer, small cell lung cancer, small intestine cancer, squamous neck cancer, testicular cancer, thymoma, thyroid cancer, uterine cancer, vaginal cancer, or vascular tumors.

20. A kit for treating a neoplasm, the kit comprising:
an oncolytic virus that expresses an ectopic antigen; and
an expression vector for genetically engineering T cells to express a complementary chimeric antigen receptor capable of recognizing the ectopic antigen.
